(19) United States
(12) Patent Application Publication
(10) Pub. No.: US 2026/0150948 A1
(43) Pub. Date: Jun. 4, 2026

(54) Shampoo-Infused Sponge

(71) Applicant: Freshape Up LLC, Wilmington, DE (US)

(72) Inventor: Andre Blalock, JR., Pennsauken, NJ (US)

(21) Appl. No.: 18/965,055

(22) Filed: Dec. 2, 2024

(51) Int. Cl. A45D 34/04; A45D 19/00; A61K 8/02; A61K 8/34; A61K 8/365; A61K 8/368; A61K 8/44; A61K 8/46; A61K 8/9794; A61Q 5/02

(52) U.S. Cl. CPC A45D 34/04; A45D 19/00; A61K 8/0208; A61K 8/345; A61K 8/365; A61K 8/368; A61K 8/442; A61K 8/463; A61K 8/9794; A61Q 5/02; A45D 2200/1018; A45D 2200/1036; A61K 2800/28; A61K 2800/87

(57) ABSTRACT

A single use shampoo-infused sponge in the form of a polyester fabric material which has been impregnated with a shampoo composition specifically formulated to be applied to the hairline or other areas of the head to facilitate shaving and edging the hairline to create clean edges and other designs.

### FIELD OF INVENTION

The present invention relates to a sponge which has been infused with a shampoo composition. More particularly, the present invention relates to a disposable sponge having a shampoo composition infused therein, which can be wetted and applied to human skin.

### BACKGROUND OF THE INVENTION

Soap and detergent compositions often are used to provide an emulsifying action for dissolving and removing dirt, grime, and applied products such as cosmetics. In addition to the chemical composition, a mechanical abrasive component usually is required to complete the cleaning process, and especially for dislodging dirt, grease, and grime from the skin. Most common mechanical abrasive components include fabric materials such as a cloth or sponge. In men's barbershops, a barber typically shampoos the customer's entire head, even when the barber only is edging certain areas of the customer's hairline. Shampooing the entire head simply to clean the hairline area results in a waste of shampoo, water, and towels. It also requires both the barber and the customer to expend extra time.

### PRIOR ART

The prior art is replete with personal care items which combine a cleaning composition with a fabric material. For example, U.S. Pat. No. 4,758,467 to Lempriere is directed to a single-use disposable impregnated dry cloth which can be presented dry to a user for use as a hair and body shampoo and wash cloth for use when showering. In U.S. Pat. No. 5,952,090 to Fan, an artificial sponge body is provided having at least one crushable liquid ball, the sponge body being made of an open-cell foamed material having at least one receiving chamber within which the liquid ball is nested. U.S. Pat. No. 6,187,728 B1 to McManus discloses a soap core having a polymeric composition coated around the core. U.S. Pat. No. 6,955,817 B2 to McAtee et al. relates to a disposable dry article which can be wetted with water and then rubbed against the skin or hair, the article being in the form of a water insoluble substrate containing a lathering surfactant and a conditioning component. And, U.S. Pat. No. 10,736,472 B1 to Gardner discloses a lathering and body scrubbing cleansing device comprised of a silicone mesh pod having a soap additive. In addition, there are numerous commercial products on the market in the forms of wipes which are pre-wetted and used for cleaning and disinfecting. One commercial product is Scrubzz which is described as an 8′×8.5″ disposable sponge of polyester construction which is infused with a no-rinse soap and no-rinse shampoo.

Despite the advancements in the prior art, a need still exists for a single use, shampoo-infused sponge primarily designed to facilitate a barber wetting the hairline of an individual in preparation of shaving and edging to create a clean and crisp hairline. Such a single use, shampoo-infused sponge should be infused or impregnated with a shampoo composition specifically formulated to effectively prepare a clean surface, remove and lift away hair products, sweat, lotion, petroleum jelly products, and any other products typically found on a hairline surface. Such a product should also be composed of a high strength, non-woven material which is durable and which can be dimensioned for ease of use.

### SUMMARY OF THE INVENTION

The invention of the present subject matter relates to a single-use, shampoo infused sponge for use by a barber to clean the surface of an individual's hairline prior to creating clean edge or other designs.

Accordingly, it is an object of the present subject matter to provide a shampoo-infused sponge wherein the sponge is constructed from a polyester fabric material.

It is another object of the present subject matter to provide a shampoo-infused sponge which is dimensioned for easy use by a barber and having a preferred size of about 10.5 cm in length, 3.5 cm in width and about 1.75 mm in thickness.

It is still another object of the present subject matter to provide a shampoo-infused sponge wherein the shampoo composition is formulated specifically to be applied to the hairline of an individual.

It is a yet another object of the present subject matter to provide a shampoo-infused sponge capable of effectively preparing a clean surface along the hairline of an individual.

It is a further object of the present subject matter to provide a shampoo-infused sponge comprising a shampoo composition formulated to remove hair products, sweat, lotion, and other products typically found on a hairline surface.

It is yet a further object of the present subject matter to provide a shampoo-infused sponge comprising a shampoo composition which is impregnated into a polyester fabric material.

It is still a further object of the present subject matter to provide a shampoo-infused sponge comprising a shampoo composition which has been dried into the sponge substrate then wrapped and packaged as a single-use item.

It is an additional object of the present subject matter to provide a shampoo-infused sponge which can be wetted easily, such as with a spray bottle, to produce a wet shampoo for application to the hairline of an individual.

These and other objects of the present subject matter are accomplished by providing a sponge in the form of a polyester fabric material which has been impregnated with a shampoo composition specifically formulated to be applied to the hairline or other areas of the head to facilitate shaving and edging the hairline to create clean edges and other designs.

### BRIEF DESCRIPTION OF DRAWINGS

FIG. 1A shows the shampoo-infused sponge of the present subject matter being wetted with a spray bottle.

FIG. 1B shows the shampoo-infused sponge of the present subject matter which has been wetted being applied to the facial hairline.

FIG. 1C shows the shampoo-infused sponge of the present subject matter which has been wetted being applied to the entire hairline.

FIG. 1D shows the resulting hairline after it has been edged.

FIG. 2 illustrates one embodiment of the preferred width, length, and height of the shampoo-infused sponge of the present subject matter.

### DETAILED DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

The terms “sponge”, “polyester fabric”, “fabric material”, “shampoo-infused fabric”, derivative terms thereof, and similar terms may be used throughout the specification and it is to be understood that such terms are interchangeable and all refer to the shampoo-infused sponge of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

The present subject matter is directed to a single use, shampoo-infused sponge comprising a high strength, non-woven material which has been infused or impregnated with a shampoo composition. The single use, shampoo-infused sponge primarily is designed to facilitate a barber wetting the hairline of an individual, followed by edging the individual's hairline to create a clean and crisp hairline. With the use of the unique single use, shampoo-infused sponge, the barber simply is able to easily and conveniently apply the shampoo just to the area that needs edging, rather than shampooing the entire head. It is to be understood that the hairline surface includes the individual's forehead, the front and back of the neck, beard, mustache, and any other special cuts or designs within the body of the hair. The application of the shampoo will ensure a squeaky-clean canvas upon which the barber can create clean edges and other designs.

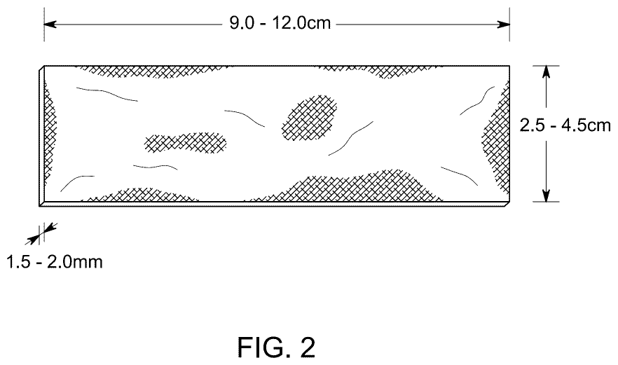

The high strength, non-woven material preferably is composed of a polyester fabric material. However, it will be understood to those skilled in the art than any high strength, non-woven material capable of being impregnated with a shampoo composition, dried, and packaged is suitable for use in the present invention. As shown in FIG. 2, the fabric material is dimensioned for ease of use, having a length of from about 9.0 cm to about 12.0 cm, a width of from about 2.5 cm to about 4.5 cm, and a thickness of from about 1.5 mm to about 2.0 mm. In a preferred embodiment, the polyester sponge has a length of about 10.5 cm, a width of about 3.5 cm and a thickness of about 1.75 mm.

The sponge is infused with a shampoo composition which has been formulated specifically to be applied to the hairline of an individual to facilitate shaving and edging the hairline. This shampoo composition effectively prepares a clean surface, removing and lifting away hair products, sweat, lotion, petroleum jelly, and other products typically found on a hairline surface.

The composition comprises from about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water. The resulting infused shampoo composition is dried into the sponge substrate then wrapped and packaged as a single-use item. A preferred shampoo composition is provided in the following example.

Shampoo Composition

| Ingredient | %/weight |
|---|---|
| Sodium Laureth Sulfate | 8.80 |
| Cocamidopropyl Betaine | 6.40 |
| Propylene Glycol | 0.48 |
| Sodium Benzoate | 0.28 |
| Phenoxyethanol | 0.24 |
| Aloe Barbadenis Leaf Extract | 0.10 |
| Parfum | 0.04 |
| Disodium EDTA | 0.02 |
| Citric Acid | 0.01 |
| Water | 83.64 |

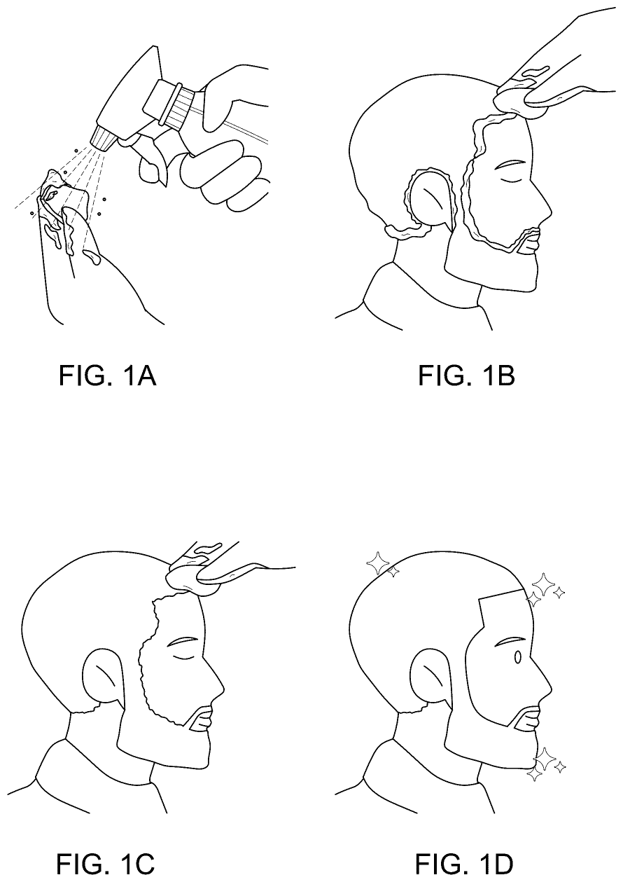

The use of the single-use shampoo-infused sponge of the present invention is illustrated in FIGS. 1A, 1B, 1C and 1D. In FIG. 1A, the shampoo-infused sponge is wetted, such as with water from a spray bottle. Wetting the sponge activates the shampoo composition impregnated within the fabric material. In FIG. 1B, the barber begins the application of the shampoo to an individual's hairline with gentle manual pressure to the individual's hairline, this pressure will result in the sponge producing sufficient lather. FIG. 1C shows the application of the shampoo along the entire hairline of the individual. Once the shampoo has been applied to the entire hairline, the barber can shave and edge up the hairline to create an ultimate sharp, very clean, crisp hairline edge that will be undeniably noticed and appreciated. Any excess shampoo can be easily wiped away with a soft cloth.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, under the provisions of 35 U.S.C § 112, sixth paragraph, unless the element is expressly recited using the phrase “means for . . . ” and no method or process step herein is to be construed under those provisions unless the step, or steps, are expressly recited using the phrase “step(s) for . . . ”

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

1. A shampoo-infused sponge comprising a high strength, non-woven fabric material and a shampoo composition which is impregnated within said fabric material in such a manner that when said fabric material is wetted, said shampoo composition is activated to produce lather, wherein said shampoo-infused sponge is dimensioned and formulated to facilitate a barber wetting and shampooing the hairline of an individual and edging the hairline to create clean edges and designs within the hairline.

2. The shampoo-infused sponge in accordance with claim 1, wherein said fabric material is composed of a polyester fabric material.

3. The shampoo-infused sponge in accordance with claim 2, wherein said fabric material is dimensioned with a length of about having a length of from about 9.0 cm to about 12.0 cm, a width of from about 2.5 cm to about 4.5 cm, and a thickness of from about 1.5 mm to about 2.0 mm.

4. The shampoo-infused sponge in accordance with claim 2, wherein said fabric material is dimensioned with a length of about 10.5 cm, a width of about 3.5 cm and a thickness of about 1.75 mm.

5. The shampoo-infused sponge in accordance with claim 2, wherein said shampoo composition is formulated specifically to be applied to the hairline of an individual to effectively prepare a clean surface by removing and lifting away hair products, petroleum jelly products, sweat, and lotion from the hairline surface.

6. The shampoo-infused sponge in accordance with claim 1, wherein said shampoo composition comprises from about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water.

7. The shampoo-infused sponge in accordance with claim 1, wherein said shampoo composition consists essentially of from about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water.

8. The shampoo-infused sponge in accordance with claim 1, wherein said shampoo composition consists of about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water.

9. The shampoo-infused sponge in accordance with claim 1, wherein said shampoo composition comprises from about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water.

10. The shampoo-infused sponge in accordance with claim 9, wherein said shampoo composition comprises:
a. about 0.10%/wt. Aloe Barbadenis Leaf Extract;
b. about 8.8%/wt. Sodium Laureth Sulfate;
c. about 6.4%/wt. Cocamidopropyl Betaine;
d. about 0.48%/wt. Propylene Glycol;
e. about 0.28%/wt. Sodium Benzoate;
f. about 0.24%/wt. Phenoxyethanol;
g. about 0.02%/wt. Disodium EDTA;
h. about 0.01%/wt. Citric Acid;
i. about 0.04%/wt. Parfum, and
j. the balance being water.

11. A shampoo-infused sponge comprising a high strength, non-woven fabric material and a shampoo composition which is impregnated within said fabric material in such a manner that when said fabric material is wetted, said shampoo composition is activated to produce lather, wherein said shampoo-infused sponge is dimensioned and formulated to facilitate a barber wetting and shampooing the hairline of an individual and edging the hairline to create clean edges and designs within the hairline, and wherein said shampoo composition comprises from about 0.05%/wt. to about 2.0%/wt. Aloe Barbadenis Leaf Extract, from about 7.5%/wt. to about 10%/wt. Sodium Laureth Sulfate, from about 5.5%/wt. to about 7.5%/wt. Cocamidopropyl Betaine, from about 0.4%/wt. to about 0.65%/wt. Propylene Glycol, from about 0.2%/wt. to about 0.35%/wt. Sodium Benzoate, from about 0.2%/wt. to about 0.3%/wt. Phenoxyethanol, from about 0.01%/wt. to about 0.03%/wt. Disodium EDTA, from about 0.005%/wt. to about 0.015%/wt. Citric Acid, from about 0.03%/wt. to about 0.05%/wt. Parfum, and the balance being water.

12. The shampoo-infused sponge in accordance with claim 11, wherein said shampoo composition comprises:
a. about 0.10%/wt. Aloe Barbadenis Leaf Extract;
b. about 8.8%/wt. Sodium Laureth Sulfate;
c. about 6.4%/wt. Cocamidopropyl Betaine;
d. about 0.48%/wt. Propylene Glycol;
e. about 0.28%/wt. Sodium Benzoate;
f. about 0.24%/wt. Phenoxyethanol;
g. about 0.02%/wt. Disodium EDTA;
h. about 0.01%/wt. Citric Acid;
i. about 0.04%/wt. Parfum, and
j. the balance being water.

13. The shampoo-infused sponge in accordance with claim 11, wherein said shampoo composition is formulated specifically to be applied to the hairline of an individual to effectively prepare a clean surface by removing and lifting away hair products, petroleum jelly products, sweat, and lotion from the hairline surface.

14. The shampoo-infused sponge in accordance with claim 11, wherein said fabric material is composed of a polyester fabric material.

15. The shampoo-infused sponge in accordance with claim 14, wherein said fabric material is dimensioned with a length of about having a length of from about 9.0 cm to about 12.0 cm, a width of from about 2.5 cm to about 4.5 cm, and a thickness of from about 1.5 mm to about 2.0 mm.

16. The shampoo-infused sponge in accordance with claim 15, wherein said fabric material is dimensioned with a length of about 10.5 cm, a width of about 3.5 cm and a thickness of about 1.75 mm.
